(19) United States
(12) Patent Application Publication
(10) Pub. No.: US 2016/0374740 A1
(43) Pub. Date: Dec. 29, 2016

(54) RETRACTABLE SCREW GUIDE

(71) Applicant: STERLING THERAPEUTICS, LLC, COLTS NECK, NJ (US)

(72) Inventor: GORDON D. DONALD, OCEANPORT, NJ (US)

(21) Appl. No.: 15/191,010

(22) Filed: Jun. 23, 2016

Related U.S. Application Data
(60) Provisional application No. 62/183,371, filed on Jun. 23, 2015.

(51) Int. Cl. A61B 17/86; A61B 17/88

(52) U.S. Cl. CPC A61B 17/864; A61B 17/8875; A61B 17/8897

(57) ABSTRACT

A surgical screw system comprising a cannulated screw, a guide wire and a driver. The screw has a head, a tubular body with a bore, and a tip opposite the head. The guide wire, shorter than the screw, is slidably disposed within the bore of the screw. The guide wire has a working end deployable beyond the tip of the screw, and the working end is sharpened to penetrate the bone and produce a pilot hole when an axial force is applied. The guide wire extends to deploy the working end beyond the tip to create the pilot hole, and fully retracts into the screw upon installation of the screw. The driver passes through the head of the screw into the bore, and applies the axial force to the guide wire. The driver may be used to drive the screw into the bone, and is removable from the screw.

### CROSS-REFERENCE TO RELATED APPLICATIONS

This application claims the benefit of the priority date of U.S. Provisional Patent Application Ser. No. 62/183,371, titled, Retractable Screw Guide, filed Jun. 23, 2015.

### BACKGROUND OF THE INVENTION

Field of the Invention

This invention relates to the field of orthopedic fixation devices, and more specifically, to retractable devices for establishing a desirable angle for screws used in orthopedic devices.

Description of the Related Art

A variety of screws are employed in connection with orthopedic surgical techniques and devices. For example, in many spinal surgeries pedicle screws are used to fix spinal implant devices by insertion of the screws into the left and right pedicles of the vertebrae. The pedicle screws can be used as anchors for rods or other connectors, for example, in spinal fusion applications. Pedicle screws can also be used to fix spinal devices such as stand-alone cervical cages directly in the disc space. Regardless of the application, it is important for the pedicle screws to be inserted at the proper angle. Similarly, other orthopedic screws require proper insertion angles for ideal fixation.

### SUMMARY OF THE INVENTION

In one or more embodiments, a system is disclosed including a cannulated screw and guide wire, wherein the guide wire is retractable into the screw at, during or after the inception of contact of the screw with bone for deployment of the screw. In another embodiment, a cannulated screw is disclosed having an integral retractable guide wire.

Currently, simple cannulated screws are typically inserted over a separately placed guide wire that is not attached to the screw. In using such devices, the user must first place the guide wire into position and then place the screw over the guide wire. Such guide wires can inadvertently be advanced, retracted or otherwise moved during placement of the screw, or become bent or kinked. In addition, after placement, guide wires may present obstacles to work around, especially when more than one guide wire is used simultaneously in a confined space.

Moreover, guide wires by themselves cannot be used to establish a desirable screw insertion angle. Known guide wires merely guide a screw to a starting position, and subsequent insertion at a desired angle depends solely on the ability of the user.

In some instances, insertion of an orthopedic screw along a desired path or angle is not easily achievable. For example, in stand-alone cervical cages, due to the small size of the working area, it is difficult to attain a screw trajectory that will engage the mid/post vertebral body.

Retractable screw guides as disclosed herein may be inserted as a unitary device whereby the guide wire and screw travel together. The cannulated screw is collinear with the guide wire contained in a bore of the screw, preventing bending or kinking of the guide wire. The guide wire retracts into the screw preventing inadvertent advancement of the guide wire. This markedly facilitates more efficient placement of the screw.

Clinical uses of the retractable screw guides include percutaneous pedicle screws, fixation screws, etc. for stand-alone cages such as cervical stand-alone cages, orthopedic or spinal fixation and fracture fixation.

### BRIEF DESCRIPTION OF THE DRAWINGS

For the purposes of illustration, there are forms shown in the drawings that are presently preferred, it being understood, however, that the invention is not limited to the precise arrangements and instrumentalities shown.

FIG. 1 is a perspective view of a device having a cannulated screw with a guide wire disposed therein according to an embodiment of the present disclosure;

FIG. 2 is a cross-sectional view of an embodiment of an end of the device of FIG. 1 taken along line A-A′;

FIG. 3 is a cross-sectional view of the device according to FIG. 1 taken along line A-A′ with a guide wire retracted within the screw according to an embodiment of the present disclosure;

FIG. 4 is a cross-sectional view of the device according to FIG. 1 taken along line A-A′ with a guide wire extended from the screw according to an embodiment of the present disclosure;

FIG. 5 is a cross-sectional view of a bore of a cannulated screw with a guide wire retracted within the bore according to an embodiment of the present disclosure;

FIG. 6 is a cross-sectional view of a bore of a cannulated screw with a guide wire extended from the opening of the bore according to an embodiment of the present disclosure; and

FIG. 7 is a view of a device in accordance with one or more embodiments partially engaged to a cervical vertebra through a cervical cage.

### DETAILED DESCRIPTION OF THE EMBODIMENTS

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that when an element is referred to as being “coupled” or “connected” to another element, it can be directly coupled or connected to the other element or intervening elements may also be present. In contrast, when an element is referred to as being “directly coupled” or “directly connected” to another element, there are no intervening elements present. Like numbers refer to like elements throughout. As used herein the term “and/or” includes any and all combinations of one or more of the associated listed items.

In addition, spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

Well-known functions or constructions may not be described in detail for brevity and/or clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein. Unless otherwise indicated or defined, all technical and scientific terms used herein have the same meaning as commonly understood by one of ordinary skill in the art to which the invention pertains. The terminology used herein is for describing particular embodiments only and is not intended to be limiting.

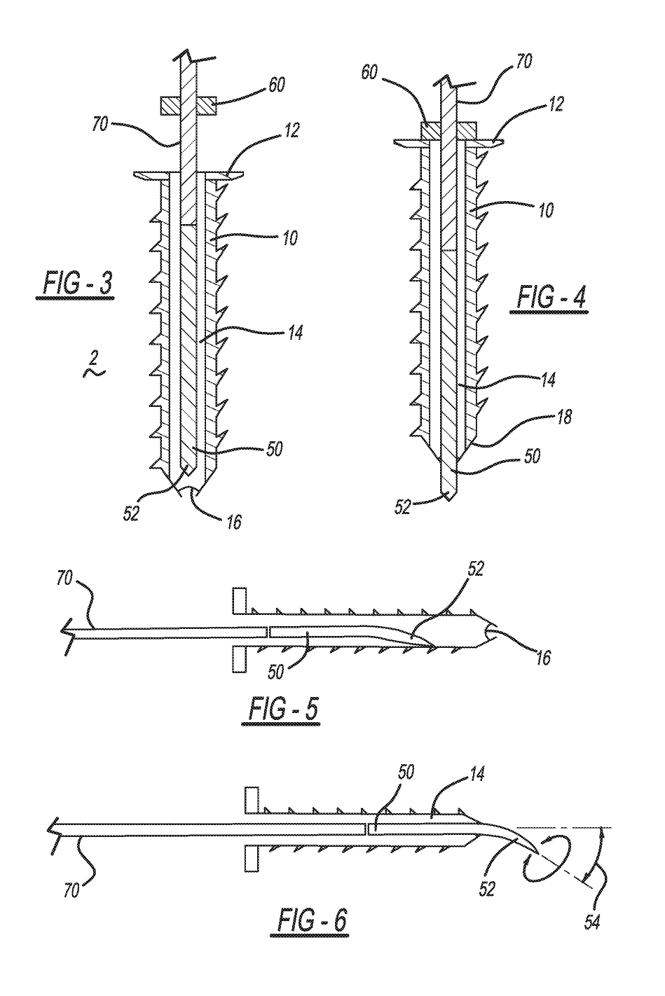

Embodiments of the present invention are described with reference to the figures. Now referring to FIGS. 1-3, a device 2 includes a cannulated screw 10 and a guide wire 50. The screw 10 includes a head 12, a bore 14 and an opening 16 distal of the head 12. The screw 10 also has a thread-starting tip 18 which tapers to a smaller diameter than the main body, to facilitate engagement of the screw 10 into the bone. The bore 14 is in communication with the opening 16 of the screw 10. A driver 70 extends through the bore 14 in the head 12 and away from the screw 10 in a direction opposite the tip 18. The driver 70 butts up against a top end of the guide wire 50. The driver 70 is used by a user/surgeon to drive the guide wire 50 out the tip 18 of the screw 10, creating a pilot hole in the target bone. As will be discussed below, the guide wire 50 can be manipulated to create the pilot hole with an orientation and angle which is desired by the surgeon for installation of the screw.

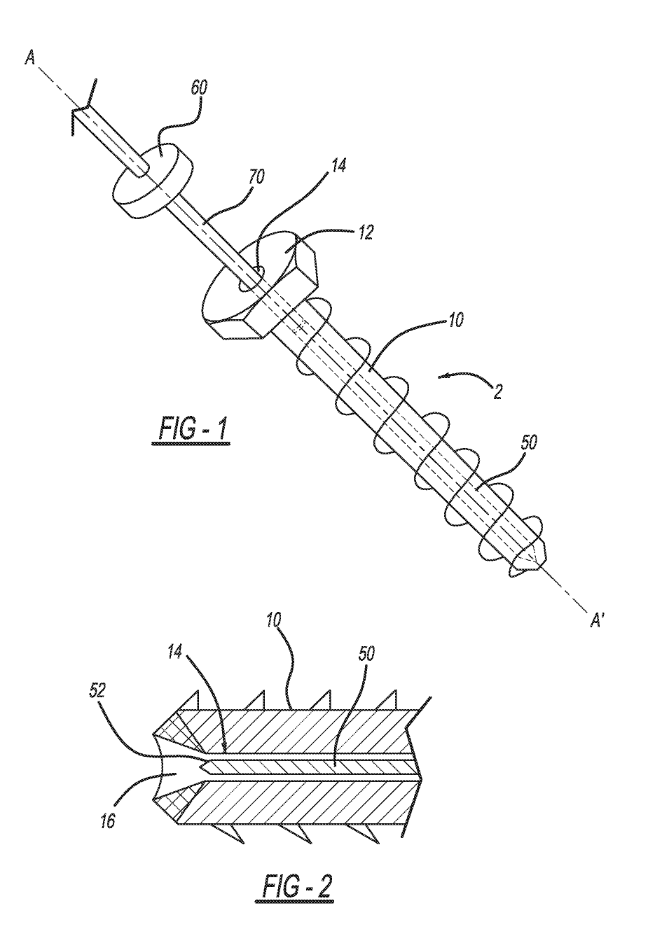

With reference to FIGS. 2 and 5, the screw opening 16 may include a conical chamfer to provide space for movement of the guide wire relative to the screw 10. The screw 10 may be any surgical screw such as but not limited to percutaneous surgical screws, fixation screws, screws used in connection with stand-alone cages, fracture fixation screws and the like. In one embodiment the screw 10 is a fixation screw having a bore 14 for a cervical stand-alone cage.

The guide wire 50 includes a working end 52. The working end 52 is operable to produce a pilot hole when pressure is applied along the long axis of the guide wire 50 in the direction of the working end 52. In one or more embodiments the working end 52 is awl- or spike-tipped. The guide wire 50 may have a diameter that is near the diameter of the bore 14 to provide a close, slidable fit therein. In other embodiments the guide wire 50 may have a diameter anywhere from 15 to 85% of the diameter of the bore 14.

The driver 70 includes a stop 60. The stop 60 limits the travel of the driver 70 into the screw 10, and thus prevents advancement of the guide wire 50 beyond a selected point. The stop 60 may be integral with the guide wire 50 or may be removably connectable. For example, the stop 60 may be a grommet of a resilient material such as rubber, the grommet having a central bore for receiving the guide wire 50. The grommet may for example be frictionally engaged to the guide wire 50 such that manual pressure can result in the advancement of the grommet along the guide wire, while the frictional engagement resists movement when force is not applied thereto. In another example, the stop 60 may be a freely slidable element, such as a disc having a central bore formed therein which is slidably engageable with the guide wire 50, the disc having a set screw to permit fixation of the stop 60 in a desired location along the guide wire 50. In other embodiments the stop 60 is not moveable. The distance the guide wire 50 may be advanced beyond the opening 16 of the screw 10 before the stop 60 on the driver 70 contacts the head 12 is a matter of design choice.

The driver 70 may also include a handle at the end opposite the screw 10, where the handle provides a better grip on the driver 70 by the user/surgeon.

The guide wire 50 is retractably disposed within the bore 14 of the screw 10. The guide wire 50 has a length which is less than the length of the screw 10, such that the guide wire 50 can be fully retracted into and contained within the body of the screw 10, both before deployment of the guide wire to create the pilot hole and after creation of the pilot hole. After the guide wire 50 is extended out the tip 18 of the screw 10 to create the pilot hole in the bone, the guide wire 50 is retracted back into the body of the screw 10 (either retracted by pulling back into the screw 10, or retracted by virtue of the advancement of the screw 10 into the pilot hole) and remains there after completion of the surgical procedure.

Now referring to FIGS. 5 and 6, the working end 52 of the guide wire 50 may be curved. The curvature of the working end 52 is such that a tip angle 54 is created, where the tip angle 54 is the angle between a tangent at the working end 52 and the straight main body portion of the guide wire 50. Different models of the guide wire 50 can be made readily available to the user/surgeon, who can select the guide wire 50 having the tip angle 54 which is needed for the particular patient's application. The tip angle 54 may preferably be in a range of 10-20°, but may be as high as 30°. Of course, the tip angle 54 is zero in straight models of the guide wire 50, as shown in FIGS. 2-4.

In use, the device 2 is initially deployed with the guide wire 50 inside the screw 10, and the driver 70 extending out of the head 12 in the direction of the user/surgeon. The screw 10 is advanced to the desired location at which the screw 10 is to be fixed to the patient. Pressure is applied to the guide wire 50 via the driver 70 such that the working end 52 drives into the bone to create the pilot hole. In the curved-tip embodiment of FIGS. 5-6, the driver 70 can be used to rotate the guide wire 50 within the bore 14 of the screw 10 so that the guide wire 50 creates the pilot hole at the angle desired by the surgeon. Establishing the position and orientation of the guide wire may be assisted by real-time images during surgery, such as ultrasound, MRI, etc.

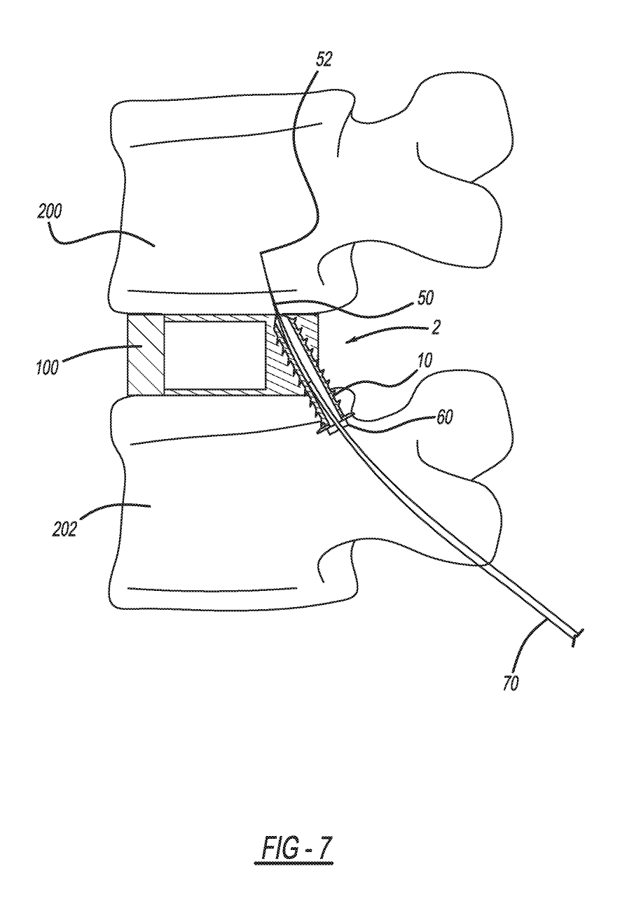

FIG. 7 depicts a cervical cage 100 disposed between adjacent vertebrae 200, 202. As discussed above, the angle of the pilot hole in the bone is determined by the user/surgeon, who can rotate the guide wire 50 in the bore 14 of the screw 10 prior to application of bone-penetrating pressure. Thus, a user can manipulate the device 2 so that the guide wire 50 is disposed through an opening formed for example in a ventral wall of the cervical cage 100 and apply force so that a pilot hole is established for example in the vertebra 200 at the desired angle for inserting the screw 10 in the bone 200. Once the pilot hole is established, the screw 10 can be advanced along the guide wire 50 and screwed into the pilot hole and the guide wire 50 can either be manually retracted, or is retracted by virtue of the advancement of the screw 10 into the bone 200 by the user. The ability to select a tip angle 54 as desired, and rotate the guide wire 50 to the desired orientation within the screw 10, gives the user complete flexibility in creating the pilot hole in the bone 200 at exactly the angle which is called for in the individual patient.

The screw 10 can be driven via external or internal driving mechanisms. Referring again to FIG. 1, the head 12 in this embodiment has an external hexagonal shape like a typical bolt head. When the pilot hole has been created in the bone by the working end 52 of the guide wire 50, and the screw 10 is ready to be driven into the bone, the driver 70 is removed from the screw 10 and a wrench-type device is used to rotate the screw 10 and drive the threads into the bone. The wrench-type device may be adapted to not only engage the hex head 12 of the screw 10 for torque, but also to apply axial force to the screw 10 to ensure positive engagement of the screw threads in the bone.

In another or overlapping embodiment, an internal driving feature is also provided in the screw 10. For example, an internal hex drive (or square, or star, or any such drive tool shape) can be included in the bore 14 inside the head 12. In this way, the external hex head 12 can be used to start the screwing of the screw 10 into the bone, then the driver 70 can be removed from the screw 10 and a hex key can be used in the internal hex pattern inside the bore 14 to drive the screw 10 fully into position. Alternately, the guide wire 50 can be removed from the screw 10 before the screw 10 is driven into the bone, and the hex key and internal hex feature can solely be used to drive the screw 10 into the bone.

In yet another embodiment, the driver 70 may be adapted to drive the screw 10 into the bone via the internal driving feature of the head 12 discussed above. For example, some or all of the driver 70 may have a cross-sectional shape matching the internal driving feature of the screw 10, such as a hex-shaped driver (“Allen wrench”). In this embodiment, the driver 70 is first used to position the screw 10 at the desired location and push the guide wire 50 out of the screw 10 to create the pilot hole. Then, the driver 70 is rotated like a screwdriver, with the hex-drive shape of the driver 70 causing the screw 10 to thread into the pilot hole in the bone. When the screw 10 is fully driven into the bone, the guide wire 50 has completely retracted into the screw 10, and the driver 70 may be removed from the head 12 of the screw 10, thus completing the installation.

Although the devices and systems of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention.

What is claimed is:

1. A surgical screw system comprising:
a cannulated screw having a head, a tubular body portion with a central bore, and a tip on an end opposite the head, where the tubular body portion has external threads suitable for threading the cannulated screw into a bone;
a guide wire slidably disposed within the central bore of the cannulated screw, said guide wire being shorter in length than the cannulated screw, said guide wire having a working end deployable through and beyond the tip of the cannulated screw, where the working end is operable to penetrate the bone and produce a pilot hole in the bone when an axial force is applied to the guide wire, and where the guide wire is extensible so that the working end is deployed beyond the tip of the cannulated screw for creation of the pilot hole and fully retractable into the cannulated screw;
a driver embodied as a long slender device, where the driver is deployed through the bore in the head of the cannulated screw to contact the guide wire at an end opposite the working end, where the driver is used to apply the axial force to the guide wire; and
a stop device coupled to the driver at a position outside the cannulated screw, said stop device being configured to establish a deployment position, where the deployment position is an axial position of the guide wire relative to the cannulated screw.

2. The system of claim 1 wherein the tip of the cannulated screw is tapered to facilitate engagement of the threads on the cannulated screw with the pilot hole in the bone.

3. The system of claim 1 wherein the guide wire is straight at the working end.

4. The system of claim 1 wherein the working end of the guide wire is curved to a specified tip angle relative to a centerline of the guide wire, and the guide wire is rotatable about its centerline within the bore of the cannulated screw, thus enabling a user to establish an approach angle of the pilot hole in the bone when pressing the working end of the guide wire into the bone with the driver.

5. The system of claim 4 wherein the central bore of the body portion of the cannulated screw includes a conical chamfer opening at the tip allowing movement of the guide wire while the working end is still within the body portion.

6. The system of claim 4 wherein the tip angle is in a range of 10-20 degrees.

7. The system of claim 1 wherein the stop device is fixed to the driver in a position which limits a distance that the guide wire can extend beyond the tip of the cannulated screw to a predefined amount.

8. The system of claim 1 wherein the stop device is slidably adjustable along the driver.

9. The system of claim 1 wherein the head of the cannulated screw has a hexagonal external shape suitable for turning with a wrench.

10. The system of claim 1 wherein the central bore of the cannulated screw at the head is configured to receive an internal driving tool.

11. The system of claim 10 wherein the internal driving tool is the driver.

12. A surgical screw for installation in a bone, said surgical screw comprising a cannulated screw body and a guide wire slidably disposed within a bore of the screw body, where the screw body has a head at one end and a tip at an opposite end, and where the guide wire is shorter in length than the screw body and has a working end deployable through and beyond the tip of the screw body, and the working end is sharpened to penetrate the bone and produce a pilot hole in the bone when an axial force is applied to the guide wire, and where the guide wire is extensible so that the working end is deployed beyond the tip for creation of the pilot hole, and fully retractable into the screw body upon installation of the screw in the bone.

13. The surgical screw of claim 12 further comprising a driver embodied as a long slender device, where the driver is deployed through the bore in the head of the cannulated screw to contact the guide wire at an end opposite the working end, where the driver is slidable within the bore and suitable to apply the axial force to the guide wire.

14. The surgical screw of claim 13 wherein the driver is configured with a cross-sectional shape which engages a compatible shape in the bore in the head of the cannulated screw, where the driver is operable to turn the cannulated screw and drive the screw into the pilot hole in the bone.

15. The surgical screw of claim 13 further comprising a stop device coupled to the driver, where the stop device prescribes a maximum deployment distance of the guide wire by limiting an amount of travel of the driver into the head of the cannulated screw.

16. The surgical screw of claim 12 wherein the working end of the guide wire is straight relative to a centerline of the guide wire.

17. The surgical screw of claim 12 wherein the working end of the guide wire is curved to a specified tip angle relative to a centerline of the guide wire, and the guide wire is rotatable about its centerline within the bore of the screw body, thus enabling a user to establish a desired approach angle of the pilot hole in the bone when pressing the working end of the guide wire into the bone.

18. The surgical screw of claim 17 wherein the tip angle is in a range of 10-20 degrees.

19. A method for installing a surgical screw in a bone, said method comprising:
providing a surgical screw comprising a cannulated screw body and a guide wire slidably disposed within a bore of the screw body, where the screw body has a head at one end and a tapered tip at an opposite end, and where the guide wire has a working end operable to penetrate the bone;
inserting a driver tool into the bore in the head of the screw body;
positioning the surgical screw in a desired location for installation into the bone;
deploying the working end of the guide wire through and beyond the tip of the screw body into a position where the screw body is to enter the bone;
applying an axial force to the guide wire, using the driver tool, causing the working end to create a pilot hole at a desired orientation angle in the bone;
driving the screw body into the bone by using the driver tool to turn the head of the screw body, where the driver tool has an external shape transmitting torque to a corresponding internal shape of the bore in the head, and where the guide wire fully retracts into the screw body upon installation of the screw in the bone; and
removing the driver tool from the screw body.

20. The method of claim 19 wherein the working end of the guide wire is curved to a specified tip angle relative to a centerline of the guide wire, and the guide wire is rotatable about its centerline within the bore of the screw body, thus enabling a user to establish a desired approach angle of the pilot hole in the bone when pressing the working end of the guide wire into the bone.
